(19) United States
(12) Patent Application Publication
(10) Pub. No.: US 2025/0046826 A1
(43) Pub. Date: Feb. 6, 2025

(54) REDOX MEDIATOR ENCAPSULATION

(71) Applicant: BEFC, GRENOBLE (FR)

(72) Inventors: Sébastien SOLAN, SAINT MARTIN D'HERES (FR); Marius CESBRON, AZAT LE RIS (FR); Elise BESSAC, GRENOBLE (FR); William TORASSO, MOIRANS (FR); Jean-Francis BLOCH, GIERES (FR); Jules HAMMOND, GIERES (FR)

(21) Appl. No.: 18/718,758

(22) PCT Filed: Dec. 14, 2022
(86) PCT No.: PCT/EP2022/085838

§ 371 Date: Jun. 11, 2024

(30) Foreign Application Priority Data
Dec. 17, 2021 (EP) 21306821.6

(51) Int. Cl. H01M 4/90; H01M 4/86; H01M 4/88; H01M 8/16

(52) U.S. Cl. CPC H01M 4/9083; H01M 4/8668; H01M 4/8828; H01M 8/16

(57) ABSTRACT

Disclosed is a method for the preparation of a bio-active ink formulation to produce a printed bio-electrode, and their use into a biodegradable enzymatic fuel cell. The method for preparing a bio-active ink formulation can include the following steps: a) mixing a redox mediator/mesoporous carbon nanocomposite with a polymeric binder in water; and b) mixing an oxidoreductase to the mixture of step a) to form the bio-active ink.

### TECHNICAL FIELD OF THE INVENTION

The present invention relates to the use of a redox mediator encapsulated into a mesoporous carbon (i.e. a redox mediator/mesoporous carbon nanocomposite) for preparation of a bio-active ink formulation to produce a printed bio-electrode, and their use into a biodegradable enzymatic fuel cell.

In the description below, references in square brackets ([ ]) refer to the list of references at the end of the text.

### STATE OF THE ART

Biological fuel cells (Biofuel cells) offer an attractive means to provide eco-friendly and sustainable power to electronic devices, particularly for small portable devices for applications such as healthcare, environmental monitoring, bio-defense, etc. Given that enzyme-based fuel cells can operate using substrates that are abundant in the biological fluids and environmental effluent (i.e., glucose and oxygen) whilst exhibiting power densities that are often superior to microbial fuel cells, they offer an attractive proposition to augment or self-power miniaturized wearable or implantable devices [1,2,3]. Furthermore, paper-based devices are gaining popularity as propositions for these types of applications owing to their low mass, small form factor and flexibility, allowing them to conform to a range of different surfaces.

The core of this technology is based on a couple of redox enzymes that ensure the catalysis of the fuels. Redox enzymes have a protein structure (apoenzyme) comprising an active redox cofactor capable of transferring electrons from the substrate to the co-substrate. In the case where the redox protein is capable of exchanging these electrons with the electrode, then replacing the substrate or co-substrate, this is called a direct electronic transfer (DET) process (FIG. 1).

This phenomenon has been known for over 30 years and observed for many proteins, however, it is not always possible to observe this DET. Indeed, depending on the redox proteins studied and depending on the electrode material, direct electronic exchange is not always favored. In order to be able to observe the bio electrocatalytic phenomenon, it is then necessary to add a redox molecule (often of low molecular weight) which will act as an electronic transporter. This is mediated electron transfer (MET) and the molecule used as a “shuttle” is commonly called a redox mediator (FIG. 1).

The electron transfer mode (DET or MET) is very often inherent in the biomolecule used and in the electrode material.

There is a very wide variety of redox mediators commonly used in the development of biofuel cells. Among the best known, we find metal complexes (osmium complexes, ferrocene), derivatives of quinones, azines or viologen types.

To be a suitable candidate as a redox mediator, the redox probe must therefore meet several criteria:
The potential of this mediator must thermodynamically favor the Biocatalysis reaction. That is to say that its potential must be higher than that of the enzyme at the anode, and conversely, lower than that of the enzyme at the cathode. (FIG. 2).
Its redox system must be reversible.
The enzyme/mediator electronic transfer must be efficient to allow good electro-catalysis.
The chemical structure of the redox probe must respect a certain size in order to be able to “access” the active site of the enzyme, which is buried in the protein structure in order to be able to perform its role of mediator between the enzyme and the electrode.

The glucose oxidase (GOx) was initially widely used in the field of biotechnology, in particular biosensors. In a few years, it has taken its place in the topic of glucose/O₂ biofuel cells. Indeed, it was quickly demonstrated that it is possible to perform an anodic electrode with this enzyme using different electrode materials (carbon nanotubes, nanoparticles, graphene, nanofibers). However, to date it is still difficult to obtain a DET with this enzyme due to its size and its hardly accessible FAD/FADH2 active site.

Given the difficulty of obtaining a DET with GOx without modifying the protein, much work has focused on electron transfer using a redox mediator since the late 1980s. There is a very wide variety of redox mediators commonly used in the elaboration of biofuel cells. Among the best known, we find metal complexes (complexes osmium, ferrocene), derivatives of quinones, azines or viologen types. It should be noted that other enzymes than GOx could also require the use of a redox mediator such as the lactate oxidase or sarcosine oxidase for instance [4].

The mediation usually takes place with both species (enzyme and mediator) in aqueous solution.

In previous studies, ferrocene and derivatives [5], ferro/ferricyanides [6], and quinone derivatives [7] have found application as redox mediators. Other known mediators include methylene blue, Prussian blue, phenazines, methyl violet, and toluidine blue [8]. However there is usually a problem with such mediators: their insolubility. For example, the use of phenanthrenequinone (PQ, FIG. 3) as a redox mediator for anodic reaction is limited by a very low solubility of this molecule in water as well as in alcohols, thus it cannot be added in excess. Furthermore, even in solution, the mediation signal is lost after a few hours due to the diffusion of the mediator far from the enzyme and/or the electrode surface. The integration of this molecule in a water-based formulation is also problematic to print directly on a paper substrate. On the one hand its insolubility does not allow to obtain a good dispersion and on the other hand, in this configuration, most of the amount of PQ diffuses in the paper when printing and is lost for mediation with the enzyme. One strategy that is often described is the addition of an organic solvent such as acetonitrile (pure or in mixture with water) to increase the solubility of the mediator [9]. However, this strategy is not of interest for a bio-based fuel cell application.

Thus there is still a need to provide a novel redox mediator system, that does not have the prior art disadvantages of the prior art redox mediators due to their insolubility.

### DESCRIPTION OF THE INVENTION

Therefore the Inventors solved the technical problem by implementation of a redox/mesoporous carbon nanocomposite (which comprises a redox mediator entrapped in a mesoporous carbon structure by a melt diffusion vaporization strategy defined herein [10,11,12]) in a bio-active ink formulation to produce a printed bio-anode. The encapsulation of the mediator allows for a reservoir of mediator in the ink. During the life cycle of the biocell, a local supersaturation of the mediator around the mesopore is maintained. This allows to (i) increase by 150 times at least the enzyme/mediator ratio (compared to the use of mediator alone in the ink formulation) by using the nanocomposite as a mediator reservoir, and (ii) avoid using organic solvents which made the solution environmentally friendly. Therefore, the close environment of the mesoporous carbon is always saturated with mediator: the mediator that is always migrating to the paper (that is the support of the electrode) is always compensated by the mediator release entrapped in the mesoporous carbon. This slow and progressive release of the mediator makes the bioanode much more persistent (increase of its cycle life).

It is known to integrate an insoluble redox mediator into a mesoporous carbon structure to provide a redox mediator/mesoporous carbon nanocomposite. These nanocomposites are then used as electrode materials for the realization of bio-anode seat of glucose oxidation. The realization of this material goes through a heat treatment during which the mediator is brought beyond its melting point (T_{melting}), e.g. evaluated as ((T_{boiling}−T_{melting})/4)+T_{melting}, in the presence of mesoporous carbon [11]. The process can be divided into two phases. First, the redox mediator reaches its melting point, and starts to fill the spaces form by mesopores in the carbon structure and formed a thin layer on the available surfaces of the mesoporous carbon through a p-p interaction. Secondly, a thin layer of redox mediator is created on the available surfaces through a p-p interaction and the excess of mediator is vaporized (FIG. 4). Depending on the size of the mesopores and the specific surface area of the carbon, a greater or lesser amount of mediator can be integrated into the structure. Advantageously, the mediator's redox signal lasts for a longer time compared to formulating it as a solution in immobilization solutions. This afterglow makes it possible to observe a catalytic current over a period greater than the state of the art.

The term “redox mediator” refers to a redox mediator insoluble in water and potentially in alcohols, for example selected from the group consisting of quinones (such as 9,10-phenanthrenequinone (PQ), Naphtoquinone, 1-10 phenanthroline-5,6dione), ferrocene, viologens (such as methyl viologen), azines (such as methylene blue, methylene green, azur A, toluidine blue, and thionine).

The term “mesoporous carbon” refers to a material having pores in the range of 2-50 nm, according to the IUPAC classification of porosity. Traditional porous carbon materials, including activated carbons and carbon molecular sieves, are commonly synthesized using a pyrolysis process with appropriate carbon precursors such as coal, polymers, and carbides, with activation through potassium hydroxide or selective etching of metal ions in carbides by halogen gases [13]. The main manufacturers are: Kuraray, BASF, Cabot Norit, Jacobi, Carbons Ingevity Corporation, Dow Corning, Toyo Tanso, Wacker Chemicals, Shin-Etsu, Momentive Performance Materials, Honeywell International, Axens, CECA (Arkema), Zeolyst, Fujian Yuanli Active Carbon, Gelest, ADA-ES, Haycarb, Clariant, CHALCO, Huber. For example, the mesoporous carbon is chosen from the group consisting of carbon black (KB600JD, CAS number:1333-86-4).

The term “melt diffusion vaporization strategy” means different weight ratio mixture of a redox mediator (e.g. PQ) and mesoporous carbon heated in air (e.g. at about 240° C. for PQ) for 3 h between the melting point (T_{melting}) and boiling point (T_{boiling}) of the redox mediator (i.e. between 200° C. and 360° C. for PQ). The melted redox mediator was imbibed into the pores by capillary forces. However, only the redox mediator adsorbed on the surface of the carbon pores remained after heating owing to the p-p interaction between the redox mediator and the mesoporous carbon, whereas the redox mediator residue without the p-p interaction evaporated and was eliminated with the airflow because of the high vapor pressure of the redox mediator. Therefore, after cooling, the solidification of redox mediator formed redox mediator-confined in carbons particles with the intimate contact between them.

The term “bio-electrode” means an electrode composed of an oxidoreductase [e.g. with a redox mediator in the form of a redox mediator/mesoporous carbon nanocomposite since its active site is sometimes inaccessible, as it is located in the core of the enzyme, to allow direct electron transfer (DET)], in particular an oxidase or dehydrogenase, to oxidise or dehydrogenate the fuel (e.g glucose) and therefore produce electrons. For example, it is a bio-anode.

An object of the present invention is therefore a method for preparing a bio-active ink formulation, the method comprising the following steps:
a) mixing a redox mediator/mesoporous carbon nanocomposite with a polymeric binder in water; and
b) mixing an oxidoreductase to the mixture of step a) to form the bio-active ink.

According to a particular embodiment of the present invention, the oxidoreductase is an oxidase, for example a glucose oxidase, a lactate oxidase, a laccase, a bilirubin oxidase, etc . . . .

Another object of the present invention is therefore a method for preparing a bio-electrode, the method comprising the following steps:
a) preparing a bio-ink formulation as above described;
b) coating the bio-active ink of step a) on a substrate; and
c) drying the coated substrate of step b).

According to a particular embodiment of the method of the present invention, the polymeric binder is chosen from the group consisting of water soluble polymers, for example carboxymethyl cellulose, polyvinylalcool or polyacrylic acid.

According to a particular embodiment of the method of the present invention, the mixture of step b) comprises less than 5% of redox mediator/mesoporous carbon nanocomposite, preferably less than 3% redox mediator/mesoporous carbon nanocomposite.

According to a particular embodiment of the method of the present invention, the mixture of step b) comprises about 0.1-5%, 0.3-4%, 0.5-2% of oxidoreductase, preferably about 1% of oxidoreductase.

According to a particular embodiment of the method of the present invention, the step of coating is carried out by deposition on the substrate of a layer of the bio-active ink formulation with a thickness of 5 to 40 μm.

According to a particular embodiment of the method of the present invention, the substrate is chosen from the group consisting of carbon based material such as buckypaper (BP) or cellulosic based material such as paper. In particular the substrate is previously coated with a current collector ink on a paper substrate and dried at about 200° C., preferably at about 150° C., even more preferably at about 120° C.

According to a particular embodiment of the method of the present invention, the step of drying is carried out at a temperature from about 25° C. (room temperature) to 40° C.

Another object of the present invention is a bio-active ink formulation obtained by a method of the present invention. In particular, the bio-active ink formulation comprises a redox mediator/mesoporous carbon nanocomposite, a polymeric binder in water, and an oxidoreductase. The bio-active ink formulation can be used in an electrode material, in particular a bio-electrode.

Another object of the present invention is a bio-electrode obtained by a method of the present invention. The bio-electrode can be used into a fuel cell such as, for example, an enzymatic biofuel cell or a bacterial fuel cell. Said fuel cell can be used into a device such as, for example, an electronic device, in particular for small portable device for applications such as healthcare, environmental monitoring, biodefense, etc.. The bio-electrode can also be used in biosensor applications.

Another object of the present invention is therefore a biodegradable enzymatic fuel cell comprising a bio-electrode of the present invention, and also an electronic device comprising said biodegradable enzymatic fuel cell.

### BRIEF DESCRIPTION OF THE FIGURES

FIG. 1 represents a schematic illustration of a simple arrangement for (A) direct electron transfer (DET) and (B) mediated electron transfer (MET), between the active site of an electrochemically-active enzyme and a solid electrode, during the oxidation of a substrate.

FIG. 2 represents the potential windows of redox-mediators.

FIG. 3 represents 9,10-phenanthrenequinone structure.

FIG. 4 represents thermal treatment.

FIG. 5 represents the cyclic voltammograms showing the redox response of (A) fresh electrodes at cycle 2 (B) aged electrodes at cycle 92. (squares) anode with PQ in KB600JD (circles) anode without PQ in KB600JD (triangles) anode without PQ in KB600JD but saturated PQ in the activation liquid.

FIG. 6 represents the cyclic voltagramms showing the redox response of electrodes containing different PQ/KB600JD ratios. (crosses) anode with 20% PQ in KB600JD (circles) anode with 70% PQ in KB600JD (squares) anode with 120% PQ in KB600JD.

FIG. 7 represents cyclic voltamperometry (A) between 1 and 20 cycles (B) between 20 and 50 cycles (C) after 50 cycles.

FIG. 8 represents cyclic voltammograms showing the redox response of electrodes F62D/F64D with Bucky Paper (BP) reference at (A) cycle 2 (B) cycle 20. (circles) F64D (crosses) BP (triangles) F62D

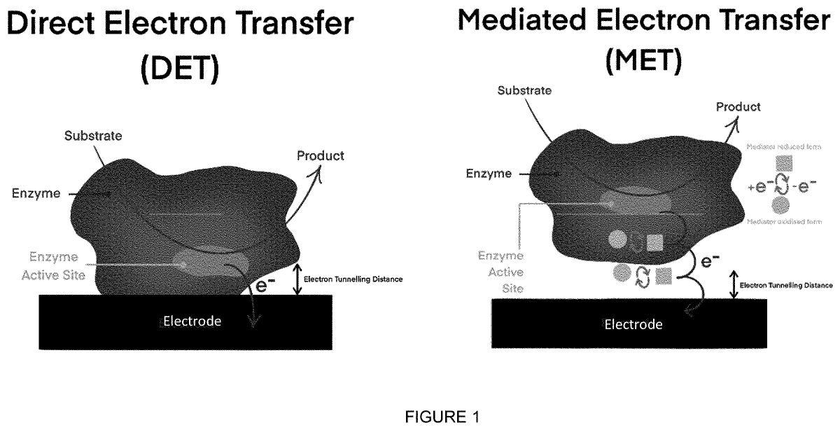

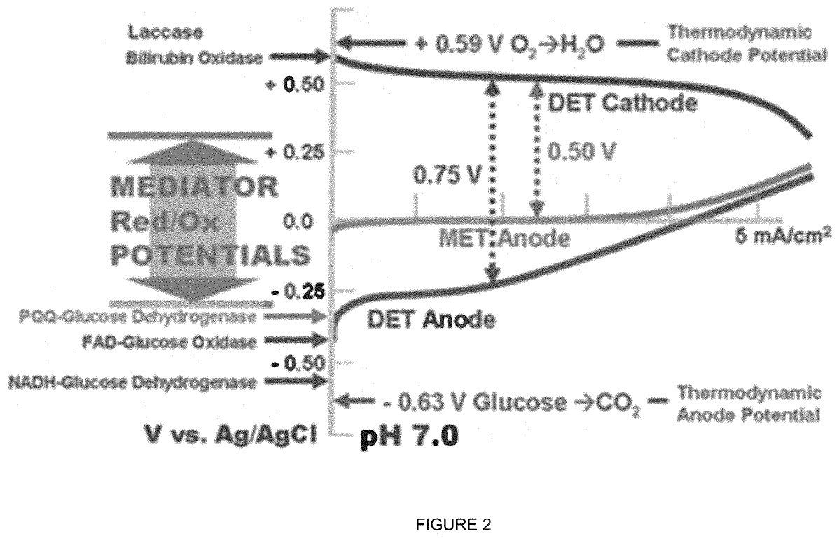

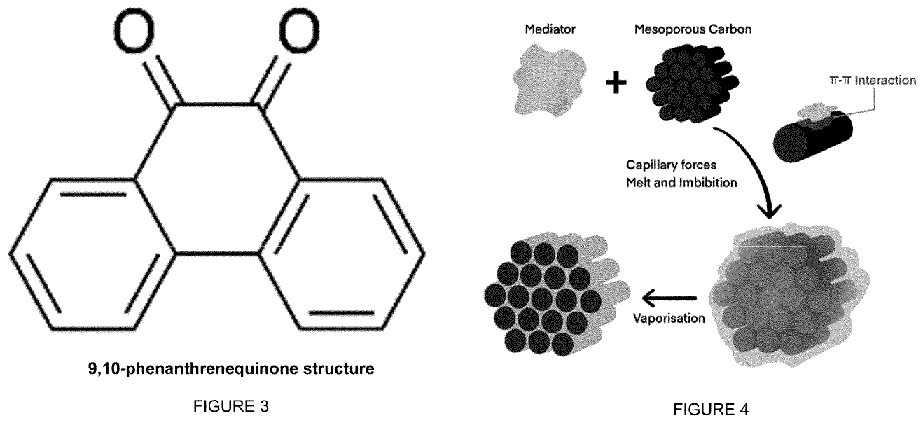

### EXAMPLES

### Example 1: Preparation of a Redox Mediator/Mesoporous Carbon Nanocomposite [9]

### Protocol Vs Selected Mediator

A 9,10-phenanthrenequinone (PQ)/mesoporous carbon (carbon black (KB600JD)) nanocomposite was prepared by following a melt-diffusion-vaporization strategy. The process temperature was set to be between the melting and the boiling point of the mediator. Experimentally, a two elevated temperature decreased the efficiency and induced a loss of material in a vapor form.

| PROPERTY OF PQ | EXPERIMENTAL AVERAGE |
|---|---|
| Melting point (° C.) | 209 |
| Boiling point°(C.) | 360 |
| Water solubility (mol/L) | 3.62e−5 |

First carbon black (KB600JD) and 9,10-phenanthrenequinone (1 g of each for 1:1 ratio) were ground together manually at lab scale or with a 3D mixer for larger amounts, pelletized manually at lab scale or with an hydraulic press for larger amounts, and then heated in an oven, at 240° C., for 3 hours in air. When the 9,10-phenanthrenequinone reached its melting point, it started to fill the spaces form by mesopores in the KB600JD carbon structure. Then, a thin layer of 9,10-phenanthrenequinone was created on the available surfaces (measurement of the specific surface area by BET method (m²/g)) of the KB600JD carbon structure through a p-p interaction, and the excess of mediator was vaporized.

Other redox mediators (e.g. ferrocene) can be charged in the same way as long as their degradation temperature is higher than their melting and boiling temperatures.

### Different Mesoporous Carbon

Four different carbons have been evaluated and loaded with the same ratio of 9,10-phenanthrenequinone (PQ).

A weight ratio mixture of PQ and mesoporous carbon (1:1) was heated in air at 240° C. for 3 h between the melting point (200° C.) and boiling point (360° C.) of PQ. The melted PQ was imbibed into the pores by capillary forces. However, only the PQ adsorbed on the surface of the carbon pores remained after heating owing to the p-p interaction between the PQ and the mesoporous carbon, whereas the PQ residue without the p-p interaction evaporated and was eliminated with the air flow because of the high vapor pressure of the PS. Therefore, after cooling, the solidification of PQ formed PQ-confined in carbons particles with the intimate contact between them.

| Mesoporous | | | | |
|---|---|---|---|---|
| carbon references | MJ(4)150-00 | KB600JD | MJ(4)010-00 | MH-00 |
| BET (m²/g) | 270 | 1270 | 1240 | 1417 |
| Mesopores size (nm) | 150 | 8 | 10 | 4 |
| Total pore | 0.35 | 1.23 | 2.10 | 1.67 |
| volume (ml/g) | | | | |

### Mass Loss Vs Mesopore Size and Temperature

The same procedure as above described was applied but by weight comparison between initial and post-treatment.

| Mesoporous | | | | |
|---|---|---|---|---|
| carbon references | MJ(4)150-00 | KB600JD | MJ(4)010-00 | MH-00 |
| Mass loss | 12% | 6% | 5% | 3% |
| at 240° C. | | | | |
| Mass loss | 33% | NA | NA | NA |
| at 250° C. | | | | |
| Mass loss | 45% | NA | NA | NA |
| at 300° C. | | | | |

Mass loss of 9,10-phenanthrenequinone (PQ) in mesoporous carbons seems to be correlated with the surface area. The less the surface was, the most of the PQ was lost. In the same way, if the temperature was too elevated or far from the melting point, mass loss increased sharply and most of the mediator was lost and escaped from the structure in the form of vapors.

### Example 2: Results Using a Redox Mediator/Mesoporous Carbon Nanocomposite

Formulation with and without 9,10-Phenanthrenequinone (PQ) in Mesoporous Carbon (Black Carbon KB600JD)

In order to test the redox mediator/mesoporous carbon nanocomposite in a bio-anode configuration, electrodes were fabricated by coating a slurry comprising the nanocomposite directly on a paper substrate.

Slurry was made by mixing the redox mediator/mesoporous carbon nanocomposite or mesoporous carbon alone with a polymeric binder in water. The binder could be carboxymethyl cellulose, polyvinylalcool or polyacrylic acid (water soluble polymers). The slurry contained 3% of redox mediator/mesoporous carbon nanocomposite (or 3% of the mesoporous carbon alone), 0.3% of polymer, 1% of Gox enzyme and 95.7% of water, and thus formed a bio-active ink formulation. The dispersion was coated on the substrate with a doctor blade in order to get a homogeneous layer that constituted the electrode after drying at 40° C.

The electrochemical characterization was made with a potentiostat in 3 electrodes configuration: Working electrode=bioanode, Counter electrode=platinum wire, and Ag/AgCl reference electrode. The catalytic current was evaluated at 0.2V in each voltammograms.

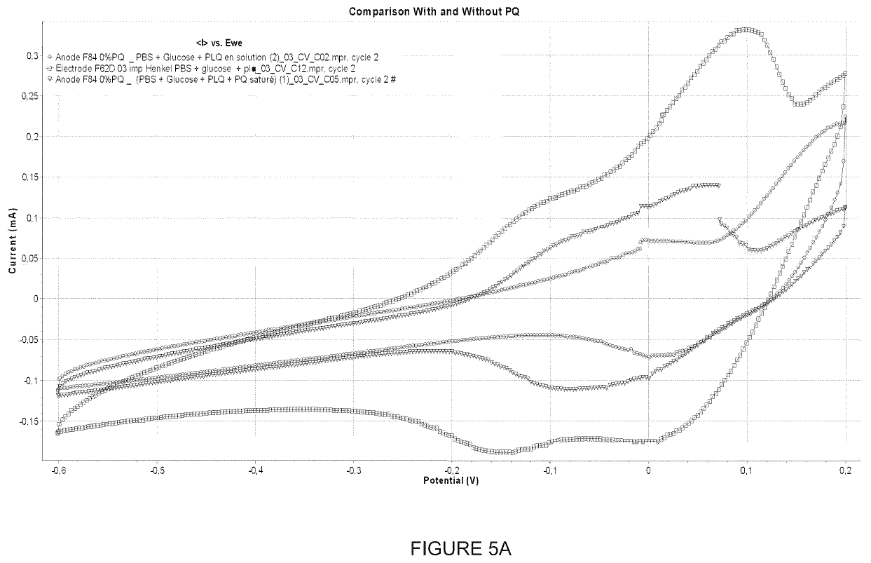

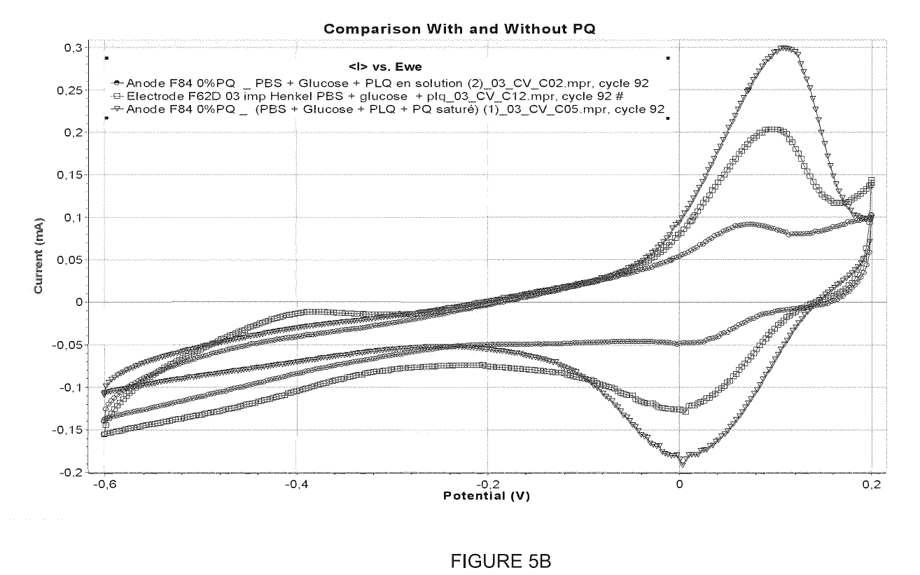

The results were represented in FIGS. 5A and 5B.

FIG. 5A represented the activity of bioanodes with fresh electrodes (at cycle 2). Cyclic voltammograms showed the redox response of electrodes containing PQ or not. Presented cyclic voltammograms were at steady-state operation (scan for 2 mV·s-1). Performance metrics were as follows:
Circles: Anode with 0% of PQ in KB600JD→Catalytic Peak current at cycle 2=0.22 mA
Squares: Anode with PQ in KB600JD→Catalytic Peak current at cycle 2=0.27 mA
Triangles: Anode with 0% of PQ in KB600JD but saturated PQ in the activation liquid (3.6*10−5M)→Catalytic Peak current at cycle 2=0.11 mA.

The results showed that mesoporous containing PQ exhibits a higher catalytic activity than mesoporous without PQ or PQ directly in the activation solution.

FIG. 5B represented the activity of bioanodes with aged electrodes (at cycle 92). Cyclic voltammograms showed the redox response of electrodes containing PQ or not. Presented cyclic voltammograms were at steady-state operation (scan for 2 mV·s-1). Performance metrics were as follows:
Circles: Anode with 0% of PQ in KB600JD. Mediator Peak current at cycle 92=0.1 mA.
Squares: Anode with PQ in KB600JD. Mediator Peak current at cycle 92=0.15 mA.
Triangles: Anode with 0% of PQ in KB600JD but saturated PQ in the activation liquid (3.6*10−5M). Mediator Peak current at cycle 92=0.10 mA.

The results showed that mesoporous containing PQ still exhibited a higher catalytic activity than mesoporous without PQ or PQ directly in the activation solution after 33 h operation time.

### Ratio KB/PQ

Several carbon-to-mediator ratios have also been performed to assess the effect of partial/complete filling of the carbon structure.

The same preparations than above were prepared but with different ratio of PQ and KB600JD as follows:

| Formulations | F81A | F64D | F82A | F62D | F83A |
|---|---|---|---|---|---|
| PQ/carbon KB600JD | 0.20 | 0.50 | 0.70 | 1.00 | 1.20 |

The electrochemical characterization was made as above described.

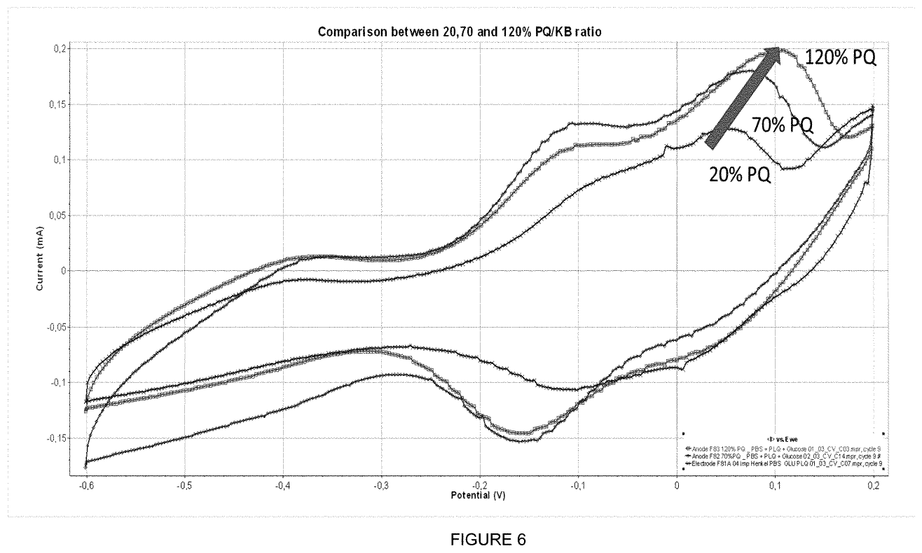

The results were represented in FIG. 6.

FIG. 6 represented cyclic voltammograms showing the redox response of electrodes containing different PQ/KB ratio. Presented cyclic voltammograms were at steady-state operation (scan for 2 mV·s-1). Performance metrics were as follows:
Crosses: Anode with 20% of PQ in KB600JD→Mediator Peak current at cycle 9=0.12 mA
Circles: Anode with 70% of PQ in KB600JD→Mediator Peak current at cycle 9=0.18 mA
Squares: Anode with 120% of PQ in KB600JD→Mediator Peak current at cycle 9=0.20 mA

As expected, a higher concentration of PQ exhibited a higher current response from the mediator.

### Example 3: Redox Signal Persistance of a Redox Mediator/Mesoporous Carbon Nanocomposite

### Comparison F64D and F62D

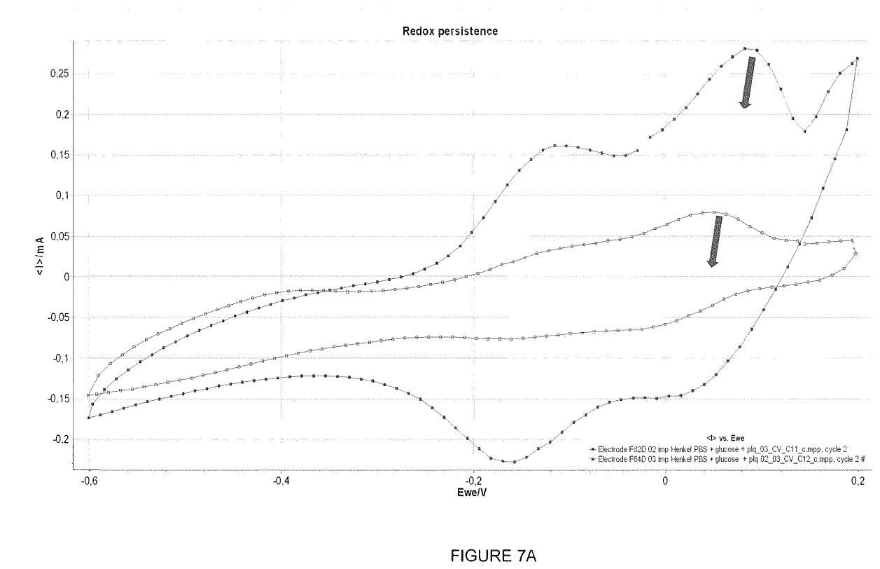

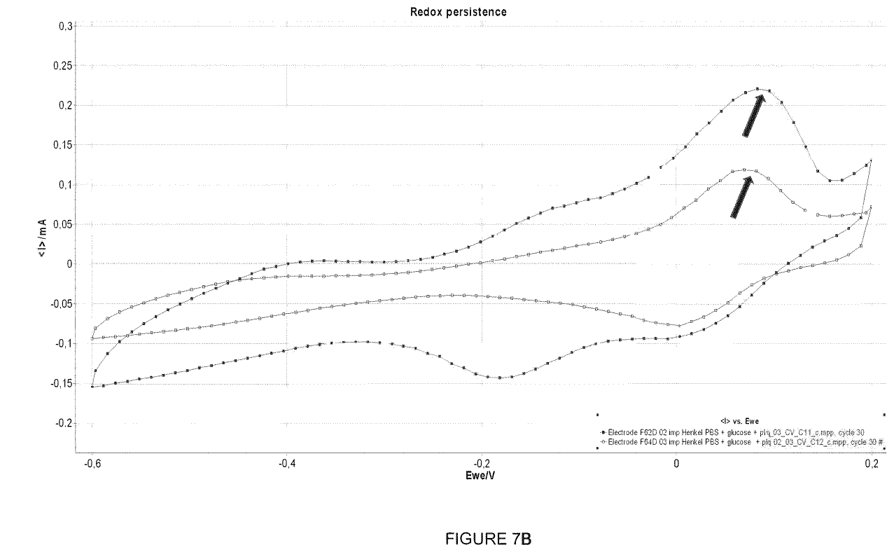

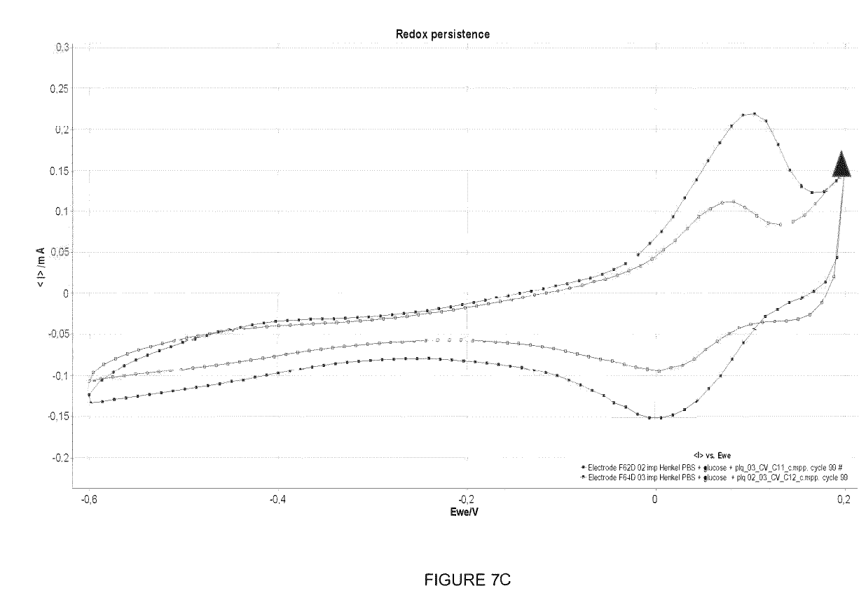

The same protocol as above described was carried out, but in this case, the evolution of both catalytic current and mediator current was followed for both formulations F64D and F62D as above defined.
Between cycles 1 and 20: decrease
   The results were represented in FIG. 7A.
Between cycles 20 and 50: increase
   The results were represented in FIG. 7B.
After cycle 50: stabilization
   The results were represented in FIG. 7C.

Advantageously, the mediator signal as well as the catalytic current stabilized after a few hours of operation for both mediator concentrations. Moreover, the F62D formulation, which had a KB/PQ ratio of 1, showed a higher catalytic current on the first cycles than the F64D formulation (KB/PQ of 0.66). From 50 cycles, both formulations showed an identical catalytic current.

Comparison F62D/F64D with BP (Bucky Paper)

Bucky paper (BP) electrodes are considered as reference according to the state of the art.

Classically, the mediators were deposited by drop casting directly on the surface of the carbon electrode. After drying, a solution containing the enzymes (glucose oxidase from Aspergillus niger, Sigma Aldrich) was also deposited by the same method and dried. The suspension used in said BP reference contained 0.25 mM of PQ and corresponded to a dry deposit of about 4.16 μg/cm².

The enzyme solution used was concentrated to 15.625 mg/ml which corresponded to a dry deposit of about 1.25 mg/cm².

The enzyme to mediator ratio was then 300.

The F62D formulation was composed of 3% of KB carbon functionalized with PQ (1:1) and 1% of enzyme. In this case, after deposition, an amount of enzyme of about 1 mg/cm² and an amount of PQ of about 1.5 mg/cm² were obtained.

The enzyme to mediator ratio was then 0.66.

For the F64D formulation, the enzyme to mediator ratio was 1.

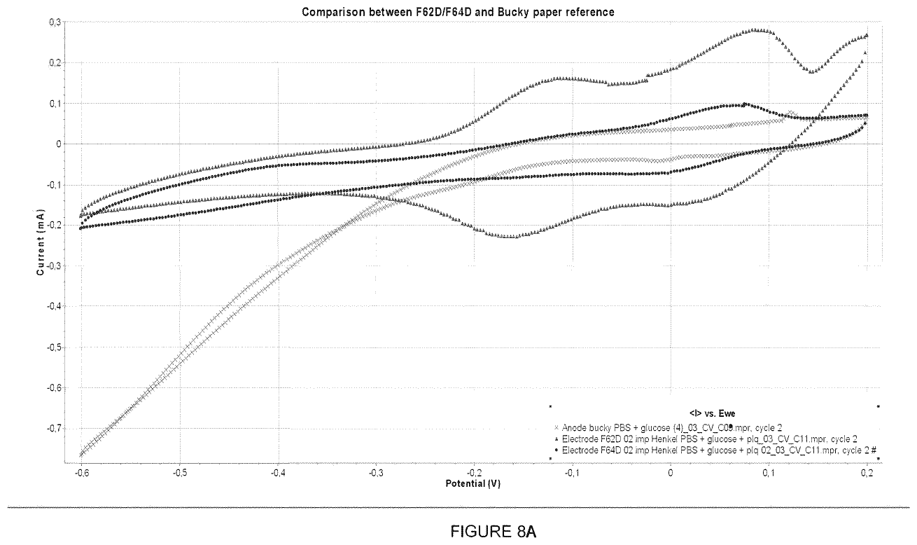

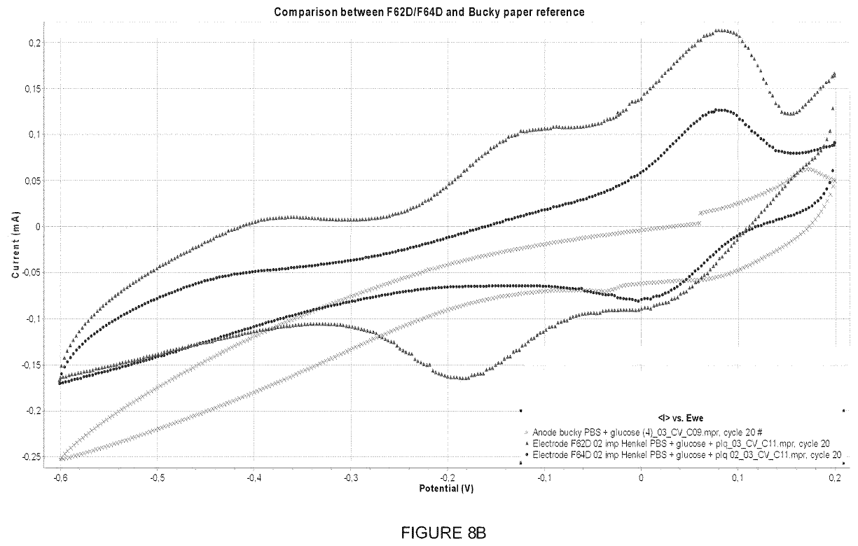

The results were represented in FIGS. 8A and 8B.

In FIGS. 8A, functionalized mesoporous carbon with 2 ratios with Bucky paper electrodes at a comparable enzyme loading were compared. FIG. 8A showed catalytic response of fresh electrodes. Presented cyclic voltammograms were at steady-state operation (scan for 2 mV·s-1). Performance metrics were as follows:
F64D (ratio 0.66): Catalytic current cycle #2=0.07 mA
F62D (ratio 1): Catalytic current cycle #2=0.27 mA
Anode Bucky paper (ratio 300): Catalytic current cycle #2=0.07 mA

In FIG. 8B, we compare functionalized mesoporous carbon with 2 ratios with Bucky paper electrodes at a comparable enzyme loading were compared. FIG. 8B showed catalytic response of aged electrodes (7 hours of activity). Performance metrics were as follows:
F64D (ratio 0.66): Catalytic current cycle #20=0.09 mA
F62D (ratio 1): Catalytic current cycle #20=0.17 mA
Anode Bucky paper (ratio 300): Catalytic current cycle #20=0.05 mA

Unlike printed anodes, the electrodes made by drop cast on the bucky paper did not have growth or stabilization phases. The catalytic signal decreased steadily.

### LIST OF REFERENCES

[1]P. Atanassov, M. Y. EI-Naggar, S. Cosnier and U. Schröder, ChemElectroChem, (2014) 1, 1702-1704
[2]E. Katz and K. MacVittie, Energy Environ. Sci., (2013) 6, 2791
[3]S. Cosnier, A. J. Gross, A. Le Goff and M. Holzinger, J. Power Sources, (2016) 325, 252-263
[4]I. Taniguchi, S. Miyamoto, S. Tomimura and F. M. Hawkridge, Mediated electron transfer of lactate oxidase and sarcosine oxidase with octacyanotungstate(IV) and octacyanomolybdate(IV), Journal of Electroanalytical Chemistry and Interfacial Electrochemistry 240 (1988) 333-339, https://doi.orq/10.1016/0022-0728(88)80335-8.
[5]A. Jędrzak, T. Rębi's, Ł Klapiszewski, J. Zdarta, G. Milczarek, T. Jesionowski, Carbon paste electrode based on functional GOx/silica-lignin system to prepare an amperometric glucose biosensor, Sens. Actuators B Chem. 256 (2018) 176-185, https://doi.orq/10.1016/i.snb.2017.10.079.
[6]D. Sun, P. Li, Q. Liu, T. Liu, M. Gu, G.-L. Wang, Versatile enzymatic assays by switching on the fluorescence of gold nanoclusters, Anal. Chim. Acta 1095 (2020) 219-225, https://doi.orq/10.1016/j.aca.2019.10.035
[7]T. Tamaki, T. Ito, T. Yamaguchi, Immobilization of hydroquinone through a space to polymer grafted on carbon black for a high-surface-area biofuel cell electrode, J. Phys. Chem. B 111 (34) (2007) 10312-10319, https://doi.org/10.1021/jp074334n10.1021/jp074334n.s001
[8]G. Rocchitta, A. Spanu, S. Babudieri, G. Latte, G. Madeddu, G. Galleri, S. Nuvoli, P. Bagella, M. I. Demartis, V. Fiore, R. Manetti, P. A. Serra, Enzyme biosensors for biomedical applications: strategies for safeguarding analytical performances in biological fluids, Sensors 16 (2016) 780, https://doi.org/10.3390/s16060780.
[9]H. Hanif, L. Ping, G. Cheng, W. Zhi-Ming, Y. Shu-Min, Y. Bing, W. Chun-Lei, M. Yu-Guang, Molecular Properties of 9,10-Phenantrenequinone and Benzil, Chem. Res. Chinese Univeristies (2009), 25(6), 950-956,
[10]C. Tian, J. Wu, Z. Ma, B. Li, X. Zhang, X. Zu, X. Xiang, S. Li, A melt-diffusion strategy for tunable sulfur loading on CC@MoS2 for lithium-sulfur batteries, Energy reports (2020) 6; suppl.7,172-180
[11]M-S. Kwon, A. Choi, Y. Park, J. y. Cheon, H. Kang, Y. N. Jo, Y-J. Kim, S. Y. Hong, S. H. Joo, C. Yang, K. T. Lee, Synthesis of ordered mesoporous phenanthrenequinone-carbon via p-p interaction-dependent vapor pressure for rechargeable batteries, Scientific reports (2014) 10; 4:7404, doi: 10.1038/srep07404
[12]U.S. Pat. No. 10,971,733
[13]A. Eftekhari, Z. Fan, Ordered mesoporous carbon and its applications for electrochemical energy storage and conversion, Mater. Chem. Front. (2017) 1, 1001-1027

1. Method for preparing a bio-active ink formulation, the method comprising the following steps:
a) mixing a redox mediator/mesoporous carbon nanocomposite with a polymeric binder in water; and
b) mixing an oxidoreductase to the mixture of step a) to form the bio-active ink.

2. Method for preparing a bio-electrode, the method comprising the following steps:
a) preparing a bio-ink formulation according to the method of claim 1;
b) coating the bio-active ink of step a) on a substrate; and
c) drying the coated substrate of step b).

3. Method according to claim 1, wherein the polymeric binder is chosen from the group consisting of carboxymethyl cellulose, polyvinylalcool or polyacrylic acid.

4. Method according to claim 1, wherein the redox mediator of the redox mediator/mesoporous carbon nanocomposite is chosen from the group consisting of quinones, ferrocene, viologens, azines.

5. Method according to claim 1, wherein the mixture of step b) comprises less than 3% redox mediator/mesoporous carbon nanocomposite.

6. Method according to claim 1, wherein the mixture of step b) comprises 0.1-5% of oxidoreductase.

7. Method according to claim 2, wherein the step of coating is carried out by deposition on the substrate of a layer of the bio-active ink formulation with a thickness of 5 to 40 μm.

8. Method according to claim 2, wherein the substrate is chosen from the group consisting of carbon-based materials or cellulosic based materials.

9. Method according to claim 8, wherein the substrate is previously coated with a current collector ink on a paper substrate and dried at 120° C.

10. Method according to claim 2, wherein the step of drying is carried out from 25° C. to 40° C.

11. Bio-active ink formulation obtained by a method of claim 1.

12. Bio-active ink formulation of claim 11, wherein it comprises a redox mediator/mesoporous carbon nanocomposite, a polymeric binder in water, and an oxidoreductase.

13. Use of the bio-active ink formulation of claim 11, as an electrode material, in particular a bio-electrode.

14. Bio-electrode obtained by a method of claim 2.

15. Biodegradable enzymatic fuel cell comprising a bio-electrode as defined in claim 14.

16. Electronic device comprising a biodegradable enzymatic fuel cell as defined in claim 15.
